(19) United States
(12) Patent Application Publication
(10) Pub. No.: US 2007/0189972 A1
(43) Pub. Date: Aug. 16, 2007

(54) Method of forming nanobubbles

(75) Inventors: Kaneo Chiba, Miyagi (JP); Masayoshi Takahashi, Ibaraki (JP)

Correspondence Address: LADAS & PARRY, 5670 WILSHIRE BOULEVARD, SUITE 2100, LOS ANGELES, CA, 90036-5679, US

(73) Assignee: REO LABORATORY CO., LTD, MIYAGI JAPAN (JP)

(21) Appl. No.: 10/591,977

(22) PCT Filed: Feb. 28, 2005
(86) PCT No.: PCT/JP05/03810

§ 371 Date: Sep. 5, 2006

(30) Foreign Application Priority Data
Mar. 5, 2004 (JP) 2004-062044

(51) Int. Cl. A61K 49/22; A01G 25/09

(52) U.S. Cl. 424/9.52; 239/1

(57) ABSTRACT

The present invention relates to a method of forming nanobubbles that have potential utility in every industrial application and that impart special functions, especially to water. The present invention is a method of forming nanobubbles by applying physical irritation to microbubbles contained in a liquid so that the microbubbles are abruptly contracted to form nanobubbles.

### TECHNICAL FIELD

The present invention relates to a method of forming nanobubbles which have potential utility in every industrial application and impart special functions, especially to water.

### BACKGROUND ART

It has been known that bubbles (microbubbles) having a diameter of not more than 50 μm have a nature different from larger bubbles and are utilized in various fields.

For example, Patent Reference 1 proposes an invention utilizing a nature of microbubbles wherein the presence of microbubbles promotes a physiological activity in creatures and increases metabolism, as a result of which ontogenetic growth is enhanced.

Recently, bubbles (bubbles having a diameter of not more than 1 μm, hereinafter referred to as nanobubbles) with a diameter smaller than that of microbubbles are said to have beneficial effects also from an industrial point of view and have become a focus of attention.

However, there is no method of forming nanobubbles. At the present state of the art, nanobubbles can momentarily exist only at the time of natural disappearance or collapse of microbubbles. Some nanobubbles with a diameter of the order of 1 μm or less can be present in a stable state by the use of a surfactant or an organic substance. Such nanobubbles, however, are encapsulated in a strong shell made up of the surfactant or organic substance, so that they are isolated from the surrounding water. These nanobubbles have not functions such as an activational effect and a bactericidal effect on organisms.

### DISCLOSURE OF THE INVENTION

The present invention has been made in view of the aforementioned circumstances and an object of the invention is to provide a method of forming nanobubbles that remain in a solution for a long time and continue to impart the solution with a function such as an activational effect or a bactericidal effect on organisms.

The present invention is directed to a method of forming nanobubbles remaining in a solution for a long time. The aforementioned object is achieved by applying physical irritation to microbubbles contained in a liquid so that the microbubbles are abruptly reduced in size.

The aforementioned object is achieved more effectively by the fact that in the step of abruptly reducing microbubbles in size, when the diameter of the microbubble is reduced to 200 nm or less, the charge density on the surface of the microbubble increases and an electrostatic repulsive force is produced, whereby the size reduction of the microbubble stops; or in the step of abruptly reducing microbubbles in size, due to ions adsorbed on the gas-liquid interface and an electrostatic attraction, both ions in the solution having opposite charges to each other and attracted to the vicinity of the interface are concentrated in a high concentration so as to serve as a shell surrounding the microbubble and inhibit dissolution of a gas within the microbubble into the solution whereby the microbubble is stabilized, or the ions adsorbed on the gas-liquid interface are hydrogen ions and hydroxide ions and electrolytic ions within the solution are used as the ions attracted to the vicinity of the interface whereby the microbubble is stabilized; or in the step of abruptly reducing microbubbles in size, the temperature within the microbubble sharply rises by adiabatic compression so that a physicochemical change in association with the ultrahigh temperature is applied around the microbubble whereby the microbubble is stabilized.

The aforementioned object is achieved more effectively when the physical irritation is to discharge static electricity through the microbubbles using a discharge device; when the physical irritation is to apply ultrasonic irradiation to the microbubbles using an ultrasonic generator; when the physical irritation is to cause a solution to flow by driving a rotor mounted in a vessel containing therein the solution and use compression, expansion and vortex flow which are produced during the flow; or when the physical irritation in the case of having a circulating circuit in the vessel to cause compression, expansion and vortex flow of the solution by passing the solution through an orifice or perforated plate having a single hole or a plurality of holes after receiving the solution that contains the microbubbles.

### BRIEF DESCRIPTION OF THE DRAWINGS

FIG. 1 shows the particle size frequency distribution of nanobubbles formed according to the methods of forming nanobubble of the present invention (even distribution: about 140 nm, standard deviation: about 30 nm);

FIG. 2 shows the relationship between the surface potential of a microbubble and the pH of an aqueous solution;

FIG. 3 shows the rise in zeta potential associated with the reduction in size of microbubbles;

FIG. 4 is a schematic view showing a mechanism wherein nanobubbles are present and stable;

FIG. 5 is a side view of an apparatus for forming nanobubbles using a discharge device;

FIG. 6 is a side view of an apparatus for forming nanobubbles using an ultrasonic generator;

FIG. 7 is a side view of an apparatus for forming nanobubbles by causing vortex flow; and

FIG. 8 is a side view of an apparatus for forming nanobubbles by causing vortex flow by a rotator.

### REFERENCE NUMERALS

1 vessel
2 discharge device
21 anode
22 cathode
3 microbubble generator
31 water inlet
32 microbubbles-contained-solution outlet
4 ultrasonic generator
5 circulating pump
6 orifice plate (perforated plate)
7 rotator

### BEST MODE FOR CARRYING OUT THE INVENTION

Nanobubbles formed by the present invention are characterized by remaining in a solution for a long time; as long as one or more months. Depending upon the nature of the gas within nanobubbles, the solution containing nanobubbles therein provides a physiological activation effect on organisms; a killing or antiproliferative effect on microorganisms such as bacteria and viruses; or a chemical reaction with an organic or inorganic substance.

The nature of nanobubbles and a method of forming nanobubbles will be described below. For illustrative convenience, descriptions are given only for the case of an aqueous solution.

The nanobubbles formed in accordance with the methods of forming nanobubbles of the present invention have a particle size or bubble diameter of not more than 200 nm. The nanobubbles formed in accordance with the methods of forming nanobubbles of the present invention remain in an aqueous solution for a long time; as long as one or more months. A preservation method of the aqueous solution containing nanobubbles therein is not particularly limited. Even when such a solution is stored in an ordinary vessel, the nanobubbles will not disappear for one or more months.

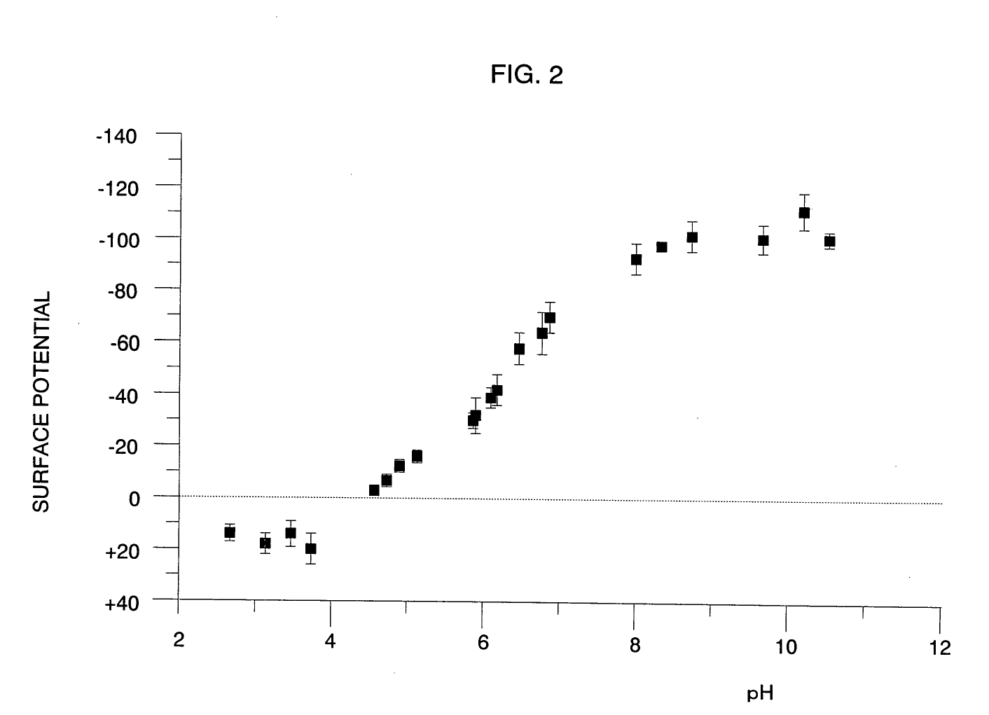

The physical property of a microbubble is to have a surface potential depending on the pH of the aqueous solution as shown in FIG. 2. This is because a hydrogen-bonding network of water at the gas-liquid interface requires more hydrogen ions and hydroxide ions as configuration factors. Since the electric charge keeps the equilibrium condition with respect to the surrounding water, it is constant regardless of the bubble diameter. Furthermore, an electrostatic force acts due to the static electrification on the surface, so that ions having the opposite electric charge are attracted to the vicinity of the gas-liquid interface.

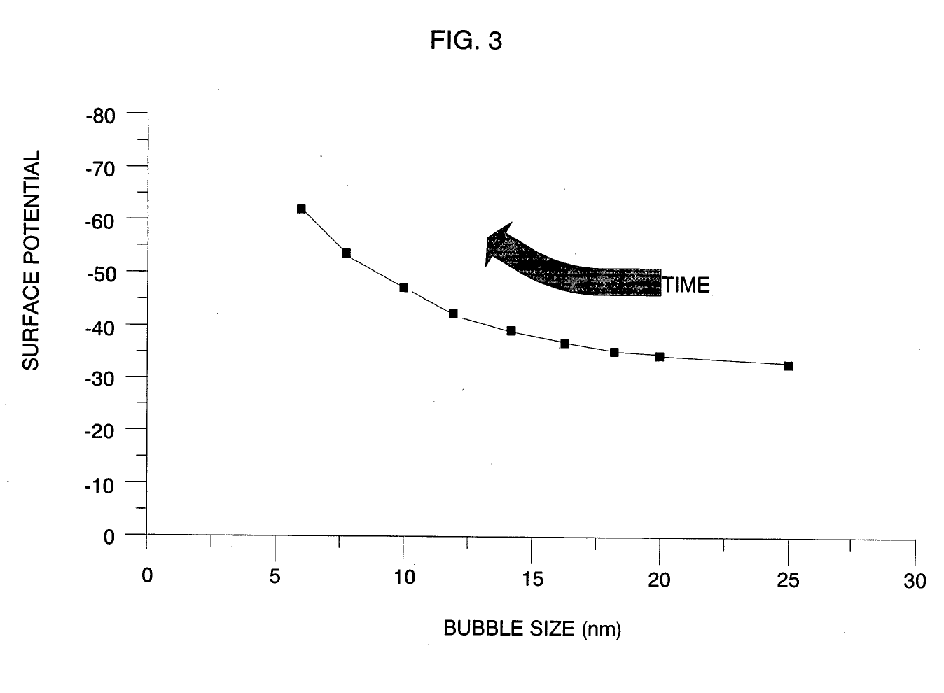

While the equilibrium charge state of a microbubble is maintained, if the microbubble is reduced in size within a short time, electric charges are concentrated. FIG. 3 shows the change of surface potential when the bubble diameter is reduced from about 25 μm to about 5 (m for 10 seconds. It can be seen from FIG. 3 that reduction in bubble diameter causes deviation from the normal equilibrium condition, which results in the concentration of the electric charges. When this size-reduction speed is made higher increased and the bubble diameter is made smaller reduced, the charge amount per unit area increases inversely with the square of the bubble diameter.

Since the microbubble is surrounded by a gas-liquid interface, the interior of the microbubble is subjected to self-pressurization under the influence of a surface tension. The pressure rise in a micro bubble with respect to an environmental pressure can be evaluated through the Young-Laplace equation.
ΔP=4σ/D (Eq. 1)

Wherein ΔP is a the pressure rise variation, σ is a the surface tension, and D is a the bubble diameter. In the case of distilled water at room temperature, for a microbubble with a diameter of 10 μm, its internal pressure rises to about 0.3 atmospheric pressures, and for a microbubble with a diameter of 1 μm, its internal pressure rises to about 3 atmospheric pressures. The gas within the self-pressurized microbubble dissolves in water according to the Henry's law. Thus, the bubble diameter is gradually reduces reduced, which increases the internal pressure of the bubble so that the bubble diameter reduction rate is accelerated. As a result, bubbles with a diameter of not more than 1 μm are completely dissolved almost instantly. That is, nanobubbles can be present only for an instant moment.

In contrast, according to the methods of forming nanobubbles of the present invention, microbubbles having a diameter of 10 μm to 50 μm are abruptly reduced by physical irritation. When the aqueous solution containing microbubbles therein is mixed with electrolytes of ferrous ions, manganese ions, calcium ions, sodium ions, magnesium ions or any other mineral ion such that the electrical conductivity in the aqueous solution containing microbubbles therein becomes not less than 300 μS/cm, the reduction in size of the bubbles is inhibited by electrostatic repulsive force. As used herein, the electrostatic repulsive force is an electrostatic force that acts on ions having the same charge and located in a diametrically opposed relationship to one another with respect to a spherical microbubble due to the increase in curvature of the sphere caused by the reduction in size of the microbubble. Since the microbubble reduced in size is subjected to pressure from surface tension, the tendency to reduce in size increases with the reduction in size of the microbubble. However, when the bubble diameter becomes smaller than 500 nm, the electrostatic repulsive force becomes evident and reduction in size of the bubble stops.

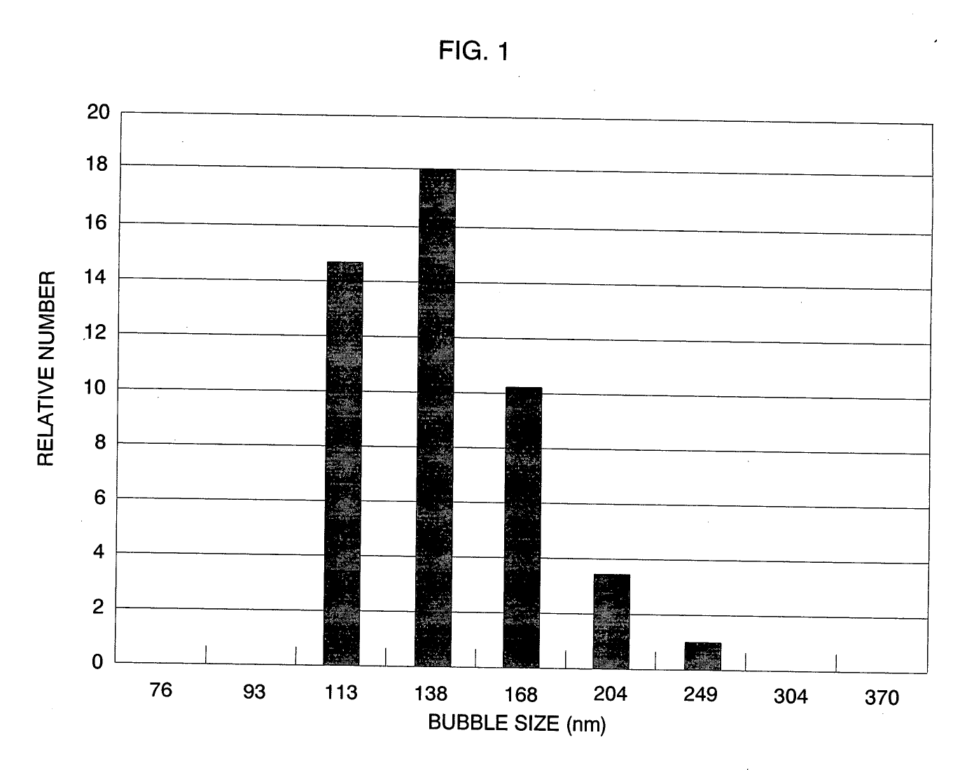

When the aqueous solution is mixed with electrolytes of ferrous ions, manganese ions, calcium ions, sodium ions, magnesium ions or any other mineral ion such that the electrical conductivity in the aqueous solution becomes not less than 300 μS/cm, the electrostatic repulsive force sufficiently acts such that the force reducing the bubble in size and the electrostatic repulsive force are balanced, as a result of which the bubble is stabilized. While the diameter of the so stabilized bubble (nanobubble diameter) differs depending upon the concentration and type of the electrolytic ion, it becomes as small as 200 nm or less as shown in FIG. 1.

The characteristics of the nanobubble are not only to keep gas therewithin in a pressurized state, but also to form a significantly strong electric field through the concentrated surface electric charges. This strong electric field exerts great influence upon the gas within the bubble and the aqueous solution around the bubble, which imparts the aqueous solution with a physiological activational effect, a bactericidal effect on organisms, chemical reactivity, etc.

FIG. 4 shows a mechanism where nanobubbles are present and stable. In the case of a nanobubble, electric charges are present on the gas-liquid interface in a significantly concentrated manner, so that the electrostatic repulsive force, which acts between the ions located in a diametrically opposed relationship to one another with respect to the sphere, inhibits the sphere (bubble) from being contracted. The concentrated high electric field serves to form an inorganic shell mainly composed of electrolytic ions such as iron around the bubble, which prevents dissipation of the gas within the bubble. This inorganic shell is different from a surfactant shell and an organic shell. Specifically, for the inorganic shell, due to the departure of the electric discharge that occurs when the nanobubble is brought into contact with other substances such as bacterium, the shell itself collapses easily. When the shell collapses, the gas within the shell is easily emitted into the aqueous solution.

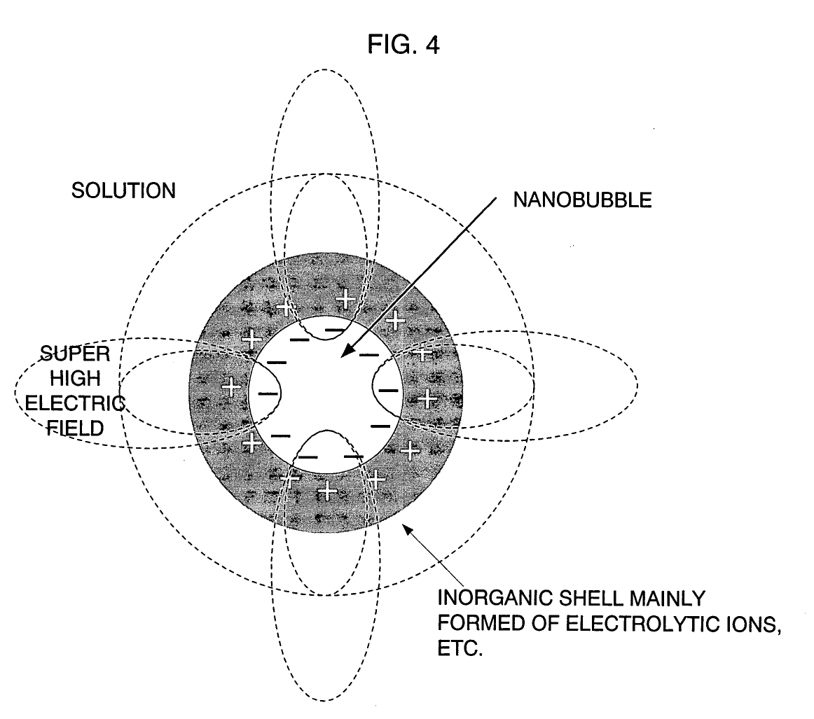

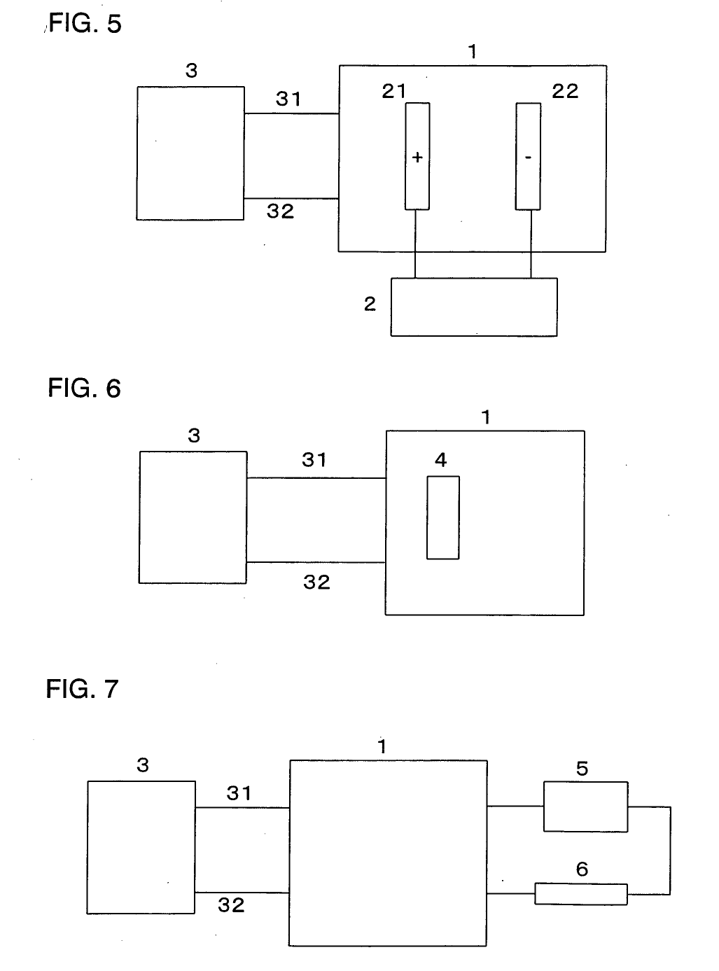

FIG. 5 is a side view of an apparatus for forming nanobubbles using a discharge device.

A microbubble generator 3 takes in an aqueous solution within a vessel 1 through a water inlet 31 and a gas is injected through a gas inlet (not shown) through which the gas for forming microbubbles within the microbubble generator 3 is injected. The gas is mixed with the aqueous solution from the water inlet 31 and microbubbles formed by the microbubble generator 3 are fed into the vessel 1 through the microbubbles-contained-solution outlet 32. As a result, microbubbles are present in the vessel 1. The vessel 1 has therein an anode 21 and a cathode 22. The anode 21 and the cathode 22 are connected to a discharge device 2.

First, using the microbubble generator 3, microbubbles are generated within the vessel 1 containing therein an aqueous solution.

Then, electrolytes of ferrous ions, manganese ions, calcium ions, or any other mineral ion is added to the aqueous solution such that the electrical conductivity in the aqueous solution becomes not less than 300 μS/cm.

Using the discharge device 2, the aqueous solution containing microbubbles therein within the vessel 1 is subjected to aqueous discharging. In order to form nanobubbles more efficiently, it is preferable that the concentration of the microbubbles within the vessel 1 have reached 50% or more of the saturated concentration. Furthermore, the voltage of the aqueous discharging is preferably in the range of 2000 V to 3000 V.

The shock wave stimulus (physical irritation) associated with the aqueous discharging reduces abruptly in size the microbubbles within the water, by which nano-level bubbles are formed. The ions existing around the bubble at this time are abruptly concentrated with the reduction in size of the bubble because the bubble reduction speed is high and there is no time for such ions to dissolve into the surrounding water. The concentrated ions produce a significantly high electric field around the bubble. Under the existence of this high electric field, hydrogen ions and hydroxide ions at the gas-liquid interface have a bonding relationship with electrolytic ions having a charge opposite thereto and located near the bubble, thereby forming an inorganic shell around the bubble. This shell inhibits spontaneous dissolution of the gas within the bubbles into the aqueous solution, so that the nanobubbles can be stably suspended in the aqueous solution. Furthermore, the nanobubble is a very tiny bubble having a diameter of not more than 200 nm, so that the nanobubble does not experience buoyant forces and rupture near the water surface, which is observed in normal bubbles.

A method of forming nanobubbles by applying ultrasound as a physical irritation to microbubbles will be described below. The same description as above is not repeated.

FIG. 6 is a side view of an apparatus for forming nanobubbles using an ultrasonic generator.

Similar to the method of forming nanobubbles by means of discharging, microbubbles are formed at a microbubble generator 3, a water inlet 31 and a microbubble-contained-solution outlet 32 and the microbubbles are fed into the vessel 1. The vessel 1 has an ultrasonic generator 4 mounted therein. The mounting position of the ultrasonic generator 4 is not particularly limited. However, in order to efficiently form nanobubbles, it is desirable to dispose the ultrasonic generator 4 between the water inlet 31 and the microbubble-contained-solution outlet 32.

First, using the microbubble generator 3, microbubbles are generated within the vessel 1 having therein water containing electrolytic ions. Then electrolytes, such as ferrite, manganese, calcium, or any other mineral is added thereto, such that the electrical conductivity in the aqueous solution becomes not less than 300 μS/cm.

Then, using the ultrasonic generator 4, ultrasound is applied to the microbubbles-contained aqueous solution within the vessel 1. In order to form nanobubbles more efficiently, it is preferable that the concentration of the microbubbles within the vessel 1 have reached 50% or more of the saturated concentration. Preferably, the oscillating frequency of the ultrasonic waves should be 20 kHz to 1 MHz and the oscillation and intermission of the application of the ultrasonic are carried out alternately at intervals of 30 seconds. However, the ultrasonic waves may be applied continuously as required.

A method of forming nanobubbles by producing vortex flow as physical irritation will be described below. The same description as above is not repeated.

FIG. 7 is a side view of an apparatus using compression, expansion and vortex flow in order to form nanobubbles. Similar to the method of forming nanobubbles by means of discharging and ultrasonic application, microbubbles are formed at a microbubble generator 3, a water inlet 31 and a microbubble-contained-solution outlet 32 and the microbubbles are fed into the vessel 1. A circulating pump 5 for regionally circulating the microbubbles-contained aqueous solution within the vessel 1 is connected to the vessel 1. An orifice plate (perforated plate) 6 having many holes is disposed within the piping (circulation piping) in which the circulating pump is provided. The orifice plate 6 is also connected with the vessel 1. The circulating pump 5 causes the microbubble-contained aqueous solution within the vessel 1 to flow the circulation piping and pass through the orifice plate (perforated plate) 6, which causes compression, expansion and vortex flow.

First, using the microbubble generator 3, microbubbles are generated within the vessel 1 having therein water containing electrolytic ions. Then, electrolytes such as ferrite, manganese, calcium, or any other mineral is added thereto such that the electrical conductivity in the aqueous solution becomes not less than 300 μS/cm.

Then, the circulating pump 5 is operated to regionally circulate the microbubbles-contained aqueous solution. The circulating pump 5 forces out the microbubbles-contained aqueous solution, which causes compression, expansion and vortex flow within the piping before and after passing through the orifice plate (perforated plate) 6. By the fact that the microbubbles are compressed or expanded when they are passed through the orifice plate and the microbubbles electrically-charged by the vortex flow produced within the piping causes an eddy-current, the microbubbles are abruptly reduced in size and stabilized as nanobubbles. The circulating pump 5 and the orifice plate (perforated plate) 6 may be arranged in the inverse order in the passage.

While a single orifice plate (perforated plate) 6 is provided in FIG. 6, a plurality of orifice plates may be provided. Furthermore, the circulating pump 5 may be omitted as appropriate. In his case, it is also possible to use a driving force of the microbubble generator 2 with respect to the aqueous solution or flowing of the aqueous solution due to a difference in elevation.

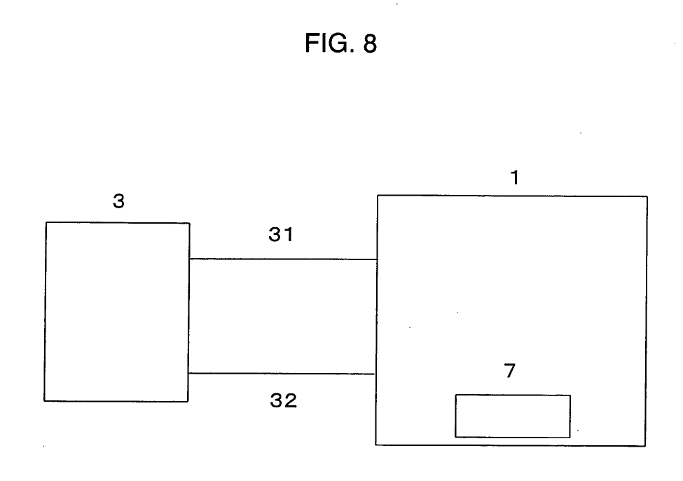

Furthermore, as shown in FIG. 8, nanobubbles may be formed by mounting in the vessel 1 a rotator 7 for producing vortex flow. Rotating the rotator 7 at 500 to 10000 rpm can efficiently produce vortex flow within the vessel 1.

While a method of forming nanobubbles according to this invention has been described, the invention is not limited thereto. For illustrative convenience, descriptions were given for the case of an aqueous solution; other solutions, such as an alcoholic solution, may be used as well as an aqueous solution.

Furthermore, oxygen, ozone or the like may be adopted as the gas from which the microbubbles are formed, which would efficiently enhance the physiological activation effect on organisms; such as a killing or antiproliferative effect on microorganisms such as bacteria and viruses, etc.

The present invention will be described in detail in connection with an example, but the invention is not limited thereto.

### EXAMPLE 1

As shown in FIG. 7, 10 liters of water containing therein electrolytic ions was placed in a vessel 1 and microbubbles were formed by a microbubble generator 3 such that the water in the vessel 1 contains microbubbles. The electrical conductivity of the aqueous solution was not less than 300 μS/cm. The microbubbles were continuously generated such that the concentration of the microbubbles within the vessel 1 reached 50% or more of the saturated concentration.

Then, the microbubble-contained aqueous solution within the vessel 1 was regionally circulated so that a part of the microbubble-contained aqueous solution was introduced into circulation piping in which a circulating pump 3 was located. The microbubble-contained aqueous solution was then introduced into the circulating pump 5 which supplied the aqueous solution to an orifice plate (perforated plate) 6 at a pressure of 0.3 MPa, wherein vortex flow was caused and the microbubbles were reduced in size to nanobubbles.

After the circulating pump 5 was operated for one hour such that a sufficient amount of nanobubbles were formed, the entire apparatus was stopped. After one week from the stop of the apparatus, the nanobubbles suspended within the vessel 1 were measured by a dynamic light scattering photometer. It was found that the nanobubbles having a medium particle diameter of about 140 nm (standard deviation of about 30 nm) remained stable.

### ADVANTAGEOUS EFFECT OF THE INVENTION

According to the method of forming nanobubbles of the present invention, it becomes possible to form nanobubbles having a bubble diameter of not more than 200 nm in a solution and causes the nanobubbles to remain in the solution for one or more months in a stable state. Furthermore, depending upon the nature of the gas within nanobubbles, the solution containing the nanobubbles therein can provide a physiological activation effect on organisms; a killing or antiproliferative effect on microorganisms such as bacteria and viruses; or a chemical reaction with an organic or inorganic substance.

### INDUSTRIAL APPLICABILITY

As described above, nanobubbles provided according to the methods of forming nanobubbles of the present invention remain in a solution for one or more months. Depending upon the nature of the gas within the nanobubbles, the nanobubbles provide a physiological activation effect on organisms; a killing or antiproliferative effect on microorganisms such as bacteria and viruses, etc. Accordingly, such nanobubbles can be applied to the medial field or the like, where sterilization and hygienic environments are required.

### LIST OF REFERENCES

Patent Reference 1: Japanese Unexamined Patent Publication No. 2002-143885

1. A method of forming nanobubbles comprising of: abruptly reducing in size microbubbles contained in a liquid having an electrical conductivity of 300 μS/cm by applying physical irritation thereto.

2. The method of forming nanobubbles according to claim 1, wherein in the step of abruptly reducing microbubbles in size, when the diameter of the microbubble is reduced to 200 nm or less, the charge density on the surface of the microbubble increases and an electrostatic repulsive force is produced, whereby the size reduction of the microbubble stops.

3. The method of forming nanobubbles according to claim 1, wherein in the step of abruptly reducing microbubbles in size, due to ions adsorbed on the gas-liquid interface and an electrostatic attraction, both ions in the solution having opposite charges to each other and attracted to the vicinity of the interface are concentrated in a high concentration so as to serve as a shell surrounding the microbubble and inhibit dissolution of a gas within the microbubble into the solution, whereby the microbubble is stabilized.

4. The method of forming nanobubbles according to claim 1, wherein the ions adsorbed on the gas-liquid interface are hydrogen ions and hydroxide ions and electrolytic ions within the solution are used as the ions attracted to the vicinity of the interface, whereby the microbubble is stabilized.

5. The method of forming nanobubbles according to claim 1, wherein in the step of abruptly reducing microbubbles in size, the temperature within the microbubble sharply rises by adiabatic compression so that a physicochemical change in association with the ultrahigh temperature is applied around the microbubble, whereby the microbubble is stabilized.

6. The method of forming nanobubbles according to claim 1, wherein the physical irritation is to discharge static electricity through the microbubbles using a discharge device.

7. The method of forming nanobubbles according to claim 1, wherein the physical irritation is to apply ultrasonic irradiation to the microbubbles using an ultrasonic generator.

8. The method of forming nanobubbles according to claim 1, wherein the physical irritation is to cause the solution to flow by driving a rotor mounted in a vessel containing therein the solution and use compression, expansion and vortex flow which are produced during the flow.

9. The method of forming nanobubbles according to claim 1, wherein in the case of having a circulating circuit in the vessel, the physical irritation is to cause compression, expansion and vortex flow of the solution by passing the solution through an orifice or perforated plate having a single hole or a lot of holes after receiving the solution in which the microbubbles are suspended.
